(19) United States
(12) Patent Application Publication
(10) Pub. No.: US 2016/0271153 A1
(43) Pub. Date: Sep. 22, 2016

(54) Novel Methods For The Treatment Of Cardiac Arrhythmias

(71) Applicant: United States Government as Represented by The Department of Veterans Affairs, Washington, DC (US)

(72) Inventors: Samuel C. Dudley, Providence, RI (US); Kai-Chien Yang, Providence, RI (US)

(21) Appl. No.: 14/547,030

(22) Filed: Nov. 18, 2014

Related U.S. Application Data
(60) Provisional application No. 61/905,788, filed on Nov. 18, 2013.

(51) Int. Cl. A61K 31/675

(52) U.S. Cl. CPC A61K 31/675

(57) ABSTRACT

The present invention relates to the therapeutic modulation of cardiac caveolin-1 to mitigate cardiac arrhythmias.

### RELATED APPLICATION

The present application claims priority to U.S. provisional patent application No. 61/905,788 filed 18 Nov. 2013, which is incorporated herein by reference in its entirety.

### GOVERNMENT RIGHTS

The present invention was made with government support under the National Institute of Health, Grant Nos. RO1 HL104025 (SCD), HL106592 (SCD), HL091071 (HHP), HL107200 (HHP), HL060678 (RDM), HL071626 (RDM); the Department of Veterans Affairs Merit Review Program, Grant Nos. BX000859 (SCD) and BX001963 (HHP); and the American Heart Association Midwest Affiliation Postdoctoral Fellowship AHA13POST14380029 (KCY). The Government has certain rights to this invention.

### FIELD

The instant disclosure relates to the therapeutic modulation of cardiac caveolin-1 to mitigate cardiac arrhythmias.

### BACKGROUND

Activation of the cardiac renin-angiotensin system (RAS) is associated with an increased risk of ventricular arrhythmia and sudden cardiac death. Increased cardiac RAS activity leads to conduction block and spontaneous ventricular arrhythmias as a result of connexin 43 (Cx43) degradation mediated by the activation of redox-sensitive tyrosine kinase c-Src signaling. The molecular mechanism of c-Src activation downstream of RAS signaling remains exclusive.

The invention described herein provides an understanding of the genetic association of caveolin 1 with arrhythmias and provides a novel approach to reduce arrhythmic risk during RAS activation.

### SUMMARY

In various embodiments, provided herein are novel approaches for reducing arrhythmic risk during renin-angiotensin system (RAS) activation in a subject in need. In specific embodiments, the method includes the regulation of Caveolin-1. In other specific embodiments, the method includes the regulation of eNOS.

In some embodiments the method includes the use of gene therapy to increase or decrease Cav1 in the subject. In some embodiments, the method includes the administration of one or more antioxidants to decrease Cav1 nitrosylation thereby decreasing arrhythmic risk. In other embodiments, the method includes the administration of an eNOS inhibitor to decrease arrhythmic risk.

### BRIEF DESCRIPTION OF THE DRAWINGS

FIGS. 1A-D demonstrate that the knockout of Cav1 leads to reduced LV conduction velocity and increases inducibility of ventricular arrhythmias, both of which are prevented by cSrc inhibition, according to one embodiment of the invention.

In FIG. 1A, representative ECG (lead II) waveforms from anesthetized adult (2-4 months) WT and Cav1^{-/-} mice are illustrated.

FIG. 1B demonstrates that the Mean±SEM PR, QRS and QTc intervals, as well as P and R wave amplitudes measured in WT (n=6) and Cav1^{-/-} (n=6) mice, were not significantly different, albeit R wave amplitudes were trending lower in Cav1^{-/-} compared to WT mice.

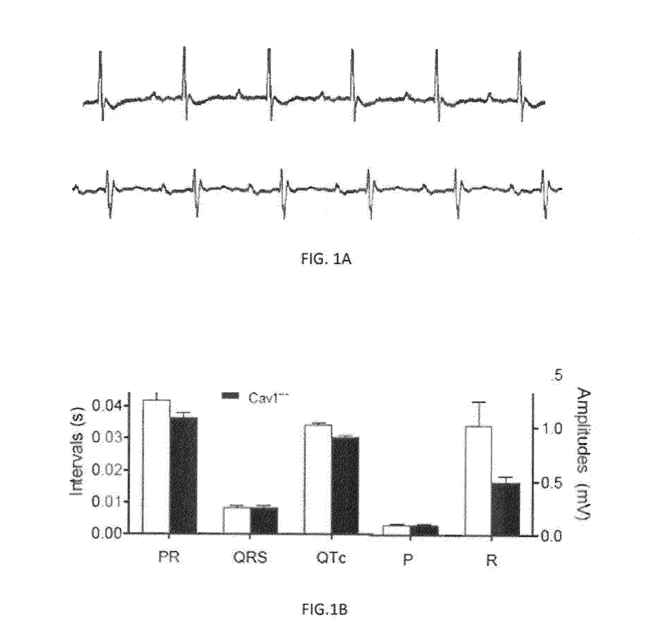

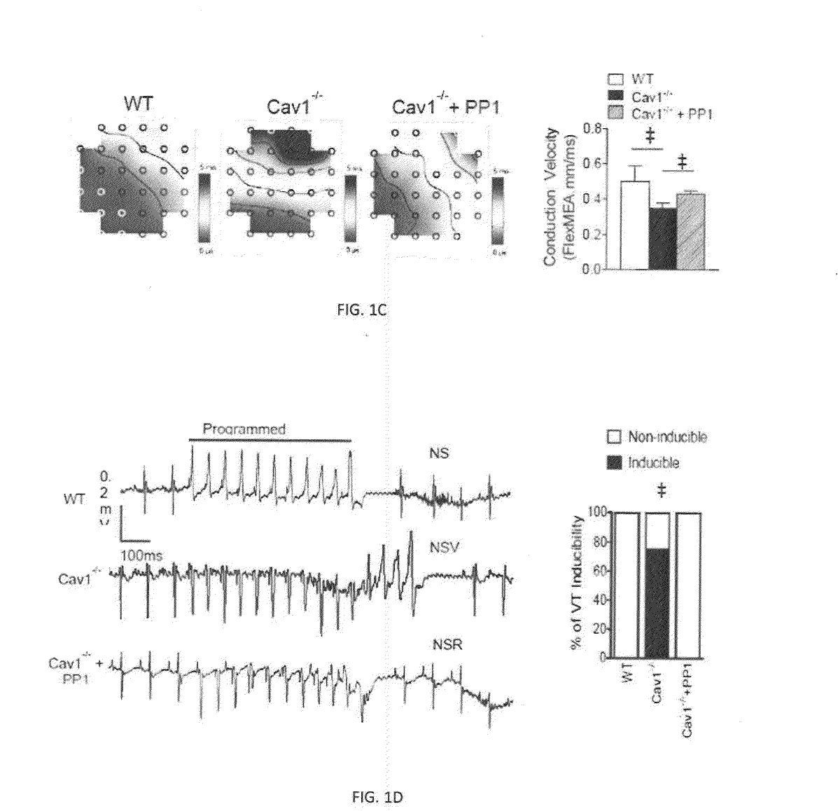

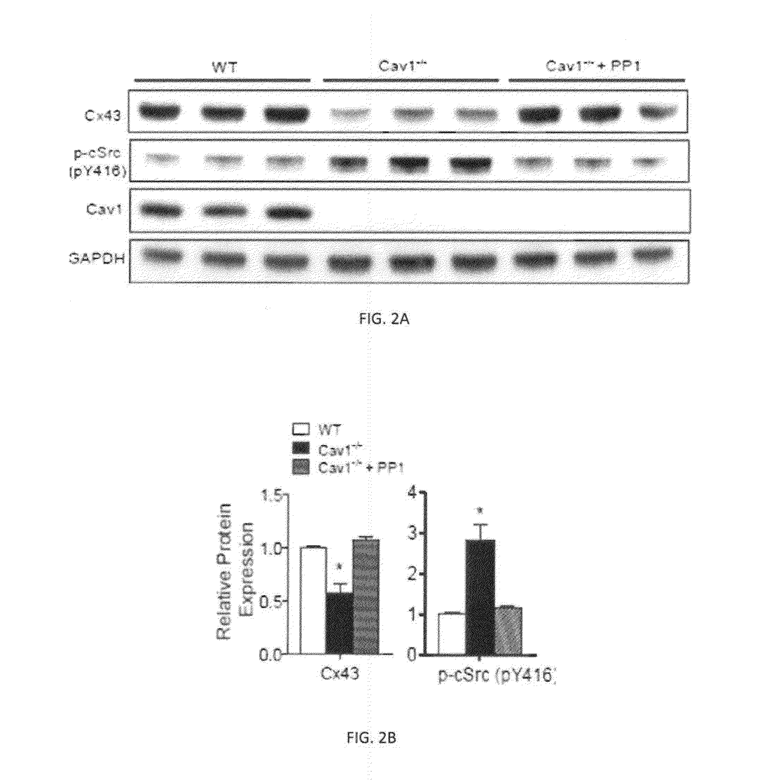

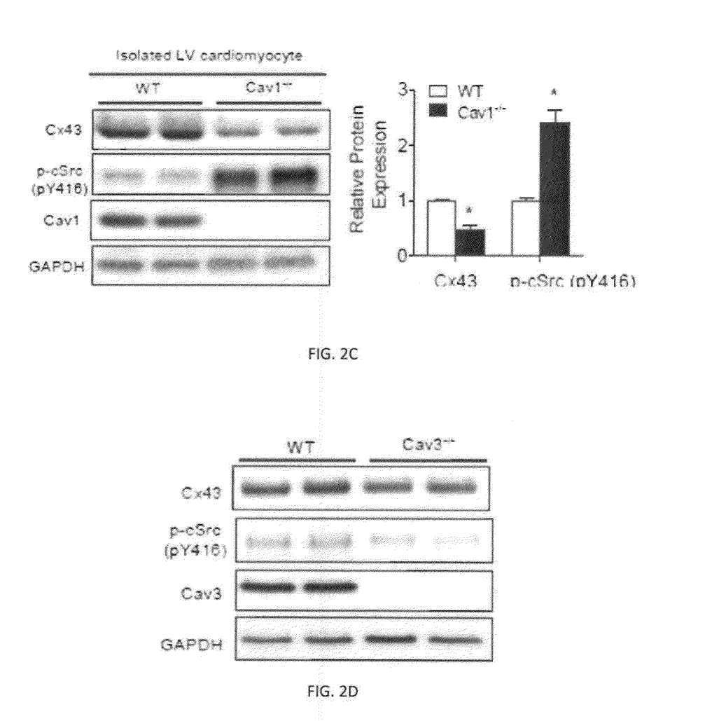

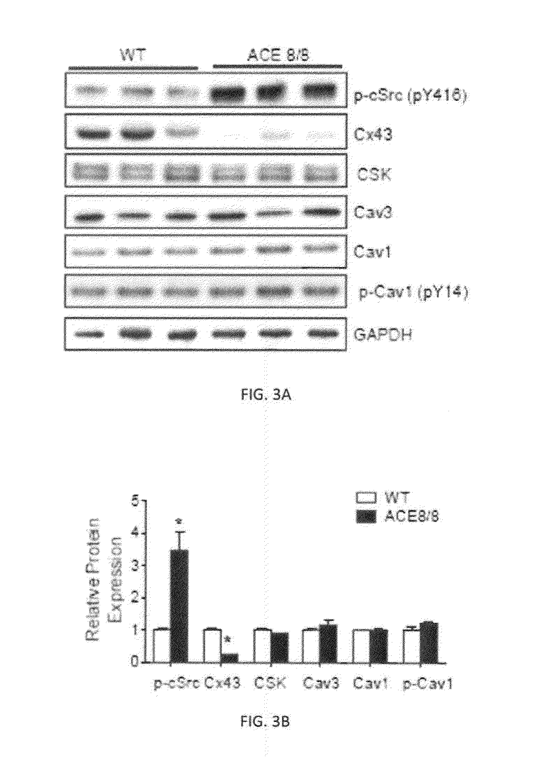

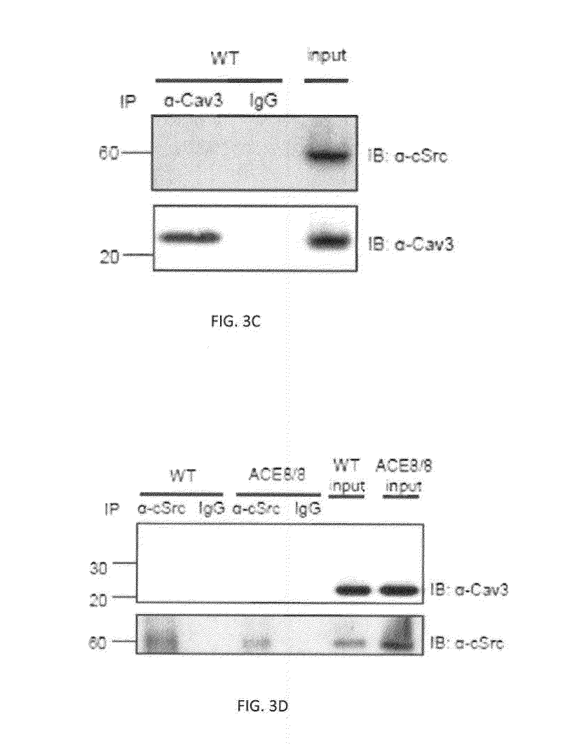

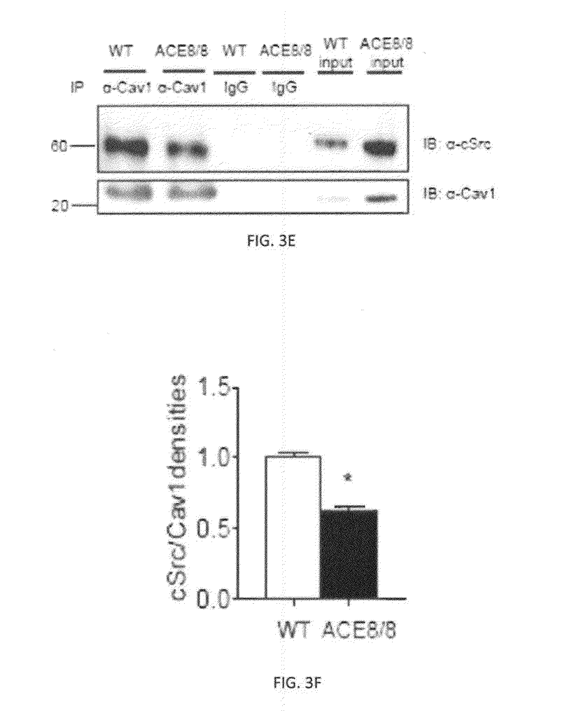

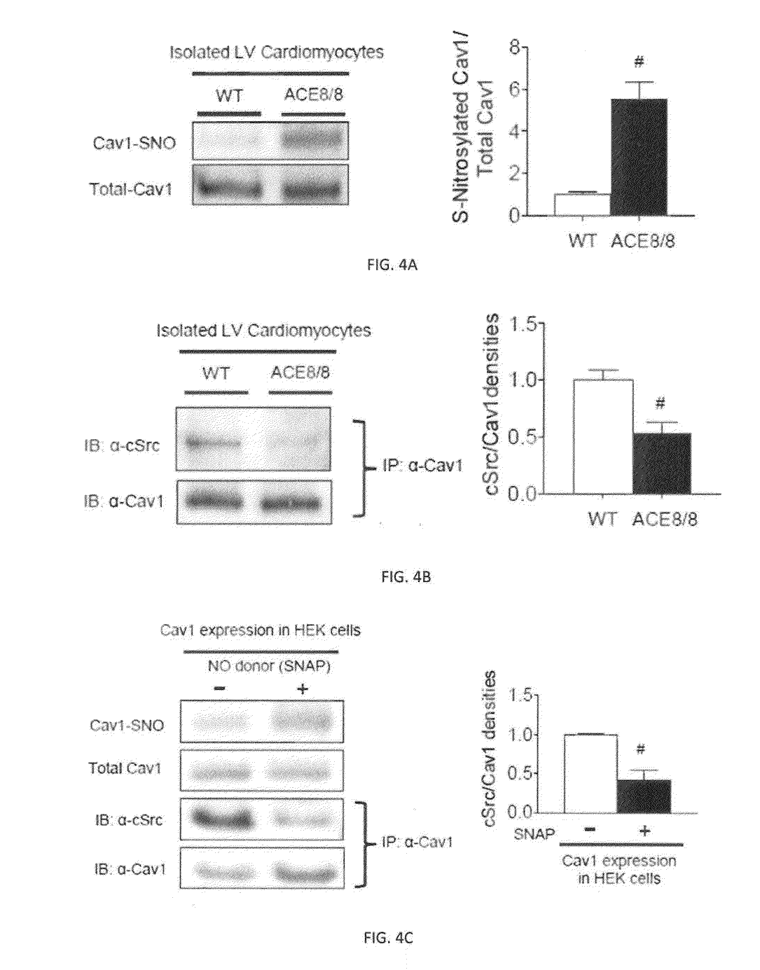

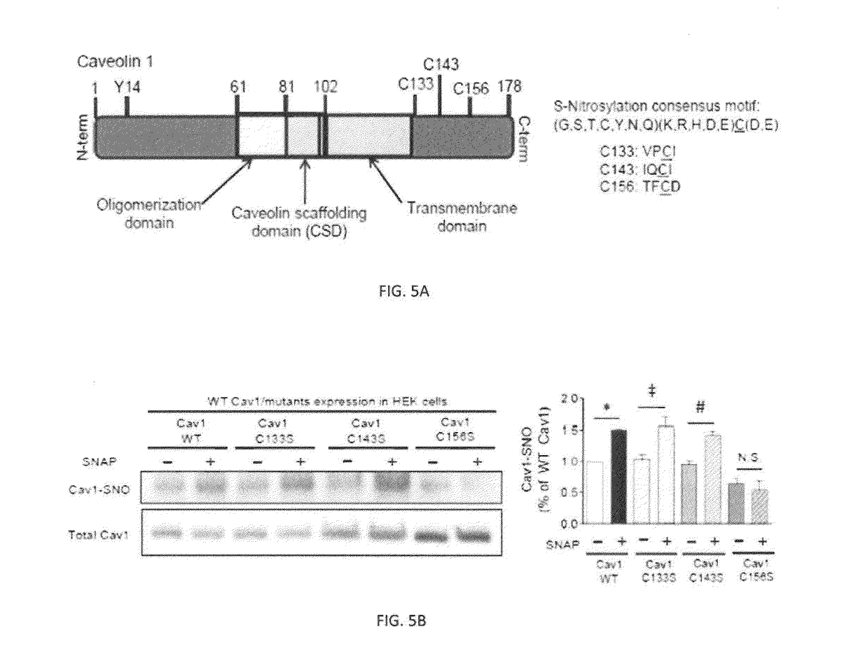

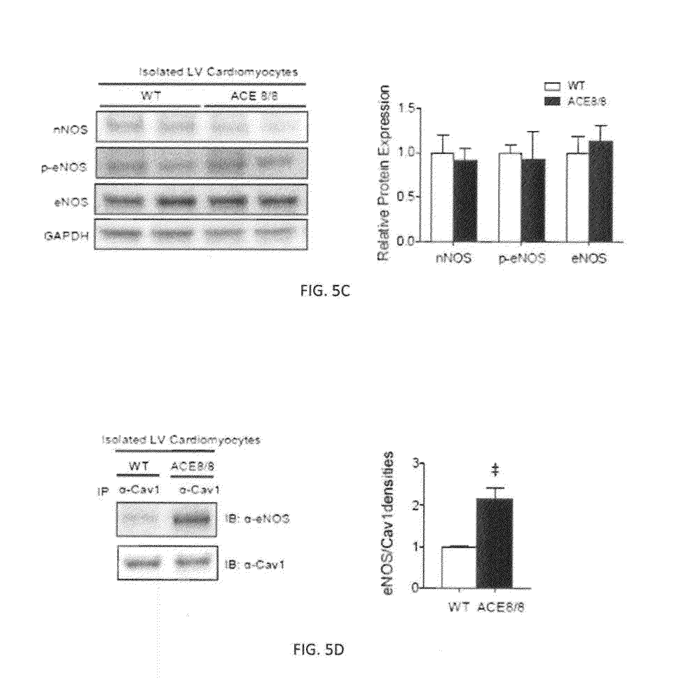

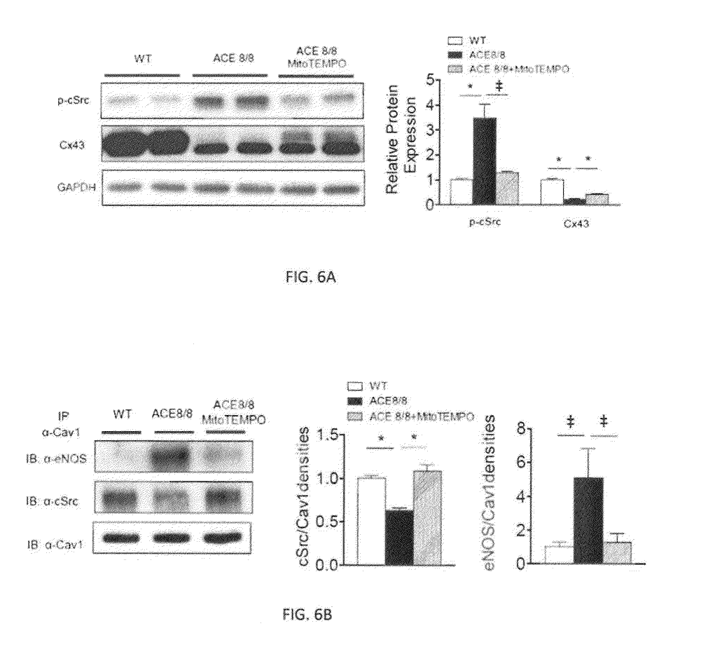

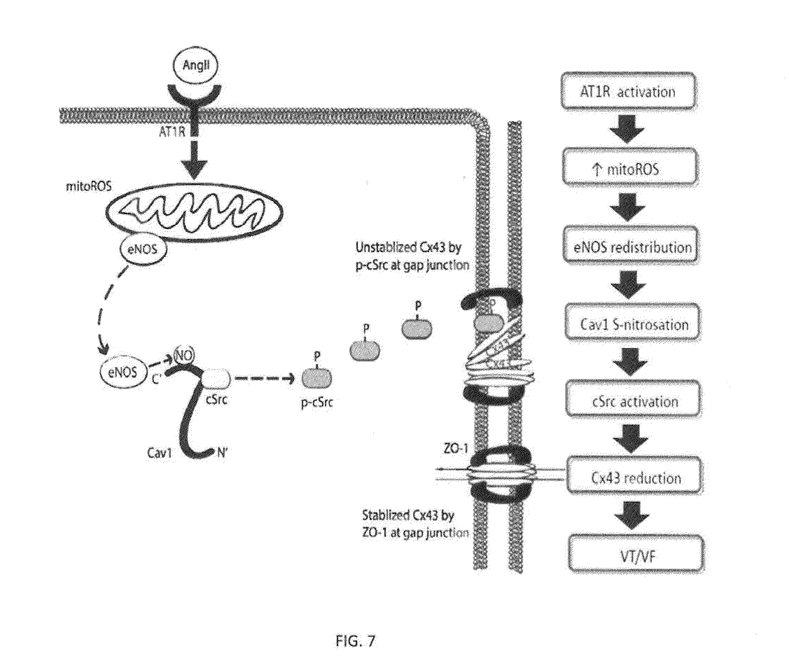

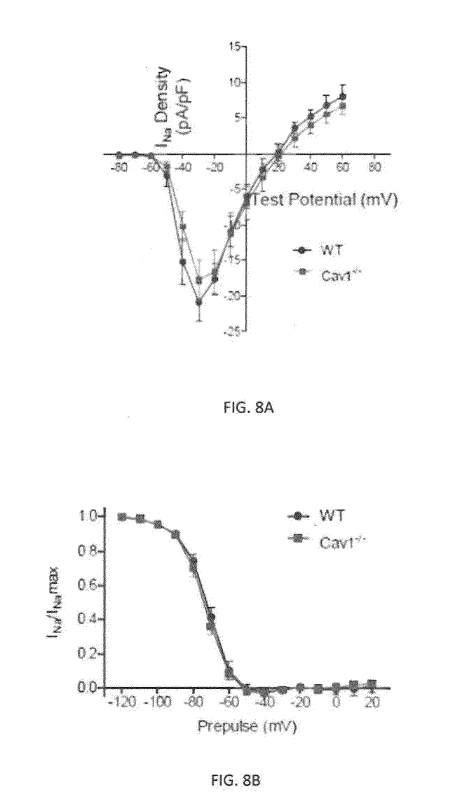

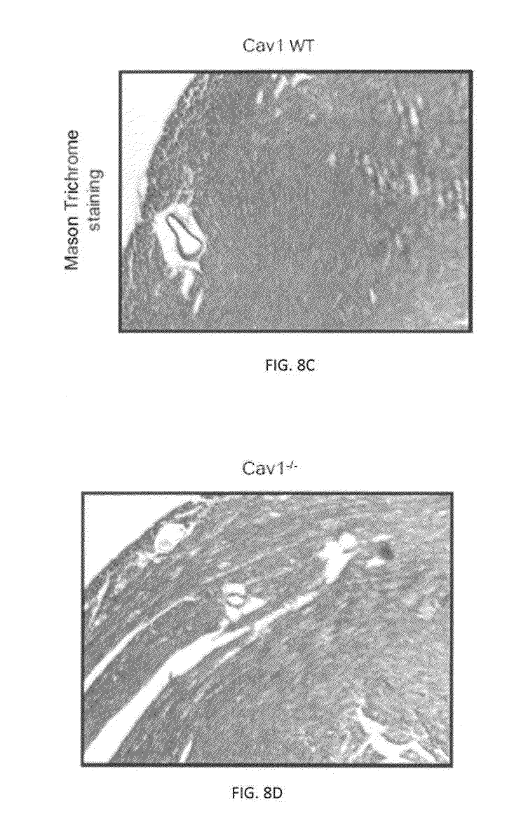

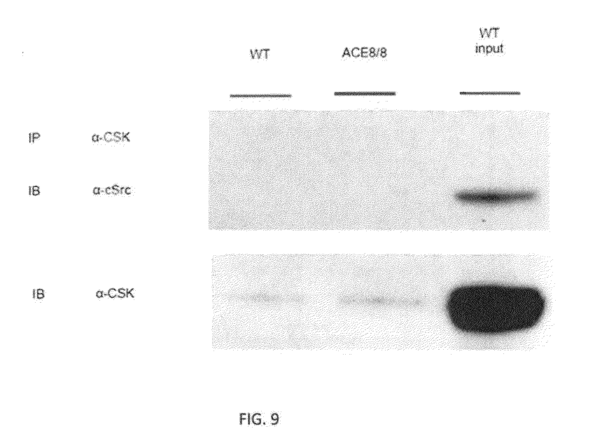

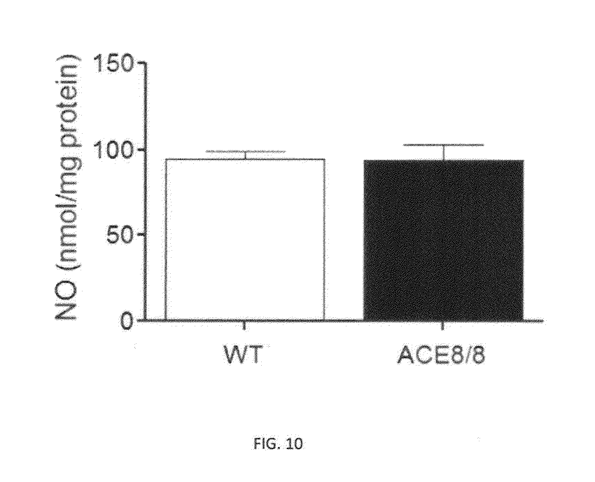

In FIG. 1C, representative LV epicardial conduction velocity recordings in WT, and Cav1^{-/-} mice treated with 4 weeks of cSrc kinase inhibitor PP1, using a 72-electrode FLEX-MEA, according to one embodiment are shown. The epicardial conduction velocity was significantly (P<0.05) reduced in Cav1−/− (n=6) compared with WT (n=6), LV. The LV conduction velocity in Cav1^{-/-} LV can be normalized with 4 weeks of PP1 treatment.

In FIG. 1D, representative surface ECG recordings from WT, Cav1^{-/-} and Cav1^{-/-} treated with PP1 during epicardial programmed electrical stimulation according to one embodiment are provided. In this embodiment, none of the WT animals were inducible for ventricular arrhythmias, whereas 75% (n=4) of Cav1^{-/-} mice were inducible for non-sustained ventricular tachycardia (NSVT) (‡P<0.05). PP1 treatment in Cav1^{-/-} mice rendered them non-inducible for ventricular arrhythmias (0% inducible, n=6) with programmed stimulation.

FIGS. 2A-D demonstrate that the loss of Cav1 results in cardiac cSrc activation and Cx43 downregulation, which can be reversed by cSrc inhibition, according to this embodiment.

FIG. 2A provides representative Western blots of the LV protein lysates from WT, Cav1^{-/-} mice and Cav1^{-/-} mice treated with 4 weeks of cSrc inhibitor PP1 (1.5 mg/kg/dose intraperitoneally, 3 times per week), according to one embodiment.

FIG. 2B illustrates that cSrc phosphorylation was significantly (*P<0.001) increased in Cav1^{-/-} (n=6) compared with WT (n=6), LV, whereas Cx43 was markedly reduced in Cav1^{-/-} LV, in this embodiment. This demonstrates that four weeks of PP1 treatments prevented cSrc phosphorylation/activation and Cx43 downregulation in Cav1^{-/-} LV (n=6).

FIG. 2C provides illustrative Western blots of the isolated LV cardiomyocytes from WT (n=4) and Cav1^{-/-} (n=4) mice confirmed markedly reduced Cx43 (by 52%, P<0.001) and increased p-cSrc (by 2.5 fold, P<0.001) in cardiomyocytes with genetic deletion of Cav1, according to one embodiment.

As illustrated in FIG. 2D, in this embodiment the p-cSrc and Cx43 protein levels were not different in WT and Cav3^{-/-} LV.

FIGS. 3A-F illustrate that cardiac RAS-induced cSrc activation and Cx43 downregulation were accompanied by a decrease in Cav1-cSrc binding, according to one embodiment.

FIG. 3A and FIG. 3B illustrate a significant increase in cSrc activation (phosphorylation at pY416) and Cx43 downregulation in ACE8/8 (n=6) compared with WT (n=6) LV (*P<0.001), according to one embodiment. In this embodiment, the protein expression levels of CSK, Cav1, Cav3, and p-Cav1 (pY14) were not different in ACE8/8 and WT LV.

As illustrated in FIG. 3C and FIG. 3D, immunoprecipitation with either Cav3 (FIG. 3C) or cSrc (FIG. 3D) antibody did not show an interaction between Cav3 and cSrc in mouse LV, in this embodiment. In contrast, cSrc co-immunoprecipitated with Cav1 in mouse LV (see, FIG. 3E and FIG. 3F), and the interaction between cSrc and Cav1 was significantly (*P<0.001) reduced (*P<0.001, by ˜50%) in ACE8/8 (n=4), compared with WT (n=4) LV, in this embodiment.

FIGS. 4A-C illustrate that RAS activation induces Cav1 S-nitrosation, resulting in Cav1-cSrc dissociation, according to one embodiment.

In FIG. 4A, Cav1 SNO was assessed using biotin-switch assay in the cardiomyocytes isolated from WT (n=4) and ACE8/8 (n=4) LV. This showed the level of Cav1 SNO was significantly (^{#}P<0.01) higher in ACE8/8 than in WT LV myocytes, in this embodiment.

In FIG. 4B, co-immunoprecipitation experiments revealed that the interaction between cSrc and Cav1 was reduced in ACE8/8 (n=4), compared with WT (n=4), LV myocytes, in this embodiment.

In FIG. 4C, HEK cells co-transfected with mouse cSrc and Cav1 cDNA were subjected to NO donor (SNAP, 20 μM, 10 min) treatment, where Cav1 SNO was increased, resulting in reduced interaction between cSrc and Cav1 (^{#}P<0.01, n=4 in each group), in this embodiment.

FIGS. 5A-D illustrate that Cav1 is nitrosated at Cys156 and Cav1 SNO upon RAS activation is associated with increased eNOS-Cav1 binding, according to this embodiment.

FIG. 5A is a schematic illustration of mouse Cav1, containing three cysteine residues (C133, C143 and C156) close to the C-terminus, among which only C156 is predicted to be nitrosated, according to this embodiment.

FIG. 5B illustrates HEK cells transfected with mouse cSrc and either WT mouse Cav1 cDNA or Cav1 containing Cys133, Cys143 or Cys156 to Ser (nitrosation-resistant) single amino acid mutation, that were subjected to SNAP treatment. In this embodiment, SNAP treatment significantly increased SNO in WT, C133S and C143S, but not in C156S, Cav1 molecule (‡P<0.05, ^{#}P<0.01, *P<0.001, n=4 in each pair), suggesting C156 is the only cysteine residue in Cav1 that can be nitrosated.

FIG. 5C is a Western blot that did not reveal significant differences in the protein expression levels of nNOS, eNOS or p-eNOS in the isolated LV myocytes from WT (n=6) and ACE8/8 (n=6) mice, in this embodiment.

FIG. 5D illustrates co-immunoprecipitation experiments demonstrated significantly (‡P<0.05) increased eNOS-Cav1 binding in ACE8/8 (n=4), compared with WT (n=4), isolated LV cardiomyocytes, according to this embodiment.

FIGS. 6A-B illustrate that Mitochondria-targeted antioxidant MitoTEMPO ameliorates cardiac RAS activation-induced cSrc activation and Cx43 downregulation through reducing Cav1-eNOS interaction and restoring Cav1-cSrc binding, according to this embodiment.

As shown in FIG. 6A, two weeks of MitoTEMPO (0.7 mg/kg/day, intraperitoneally) treatment significantly attenuated cSrc activation/phosphorylation (‡P<0.05) and Cx43 downregulation (*P<0.05) in ACE8/8 LV (n=6 in each group), according to this embodiment.

As illustrated in FIG. 6B, MitoTEMPO treatment significantly reduced Cav1-eNOS interaction (*P<0.05) and restored Cav1-cSrc binding (*P<0.001) in ACE8/8 LV (n=6 in each group), according to this embodiment.

FIG. 7 is a schematic illustrating molecular mechanisms linking RAS activation to gap junction remodeling and ventricular arrhythmias, according to one embodiment. As shown in this figure, upon RAS activation, AngII binds to the AT1 receptor, which elevates the level of mitochondrial ROS (mitoROS).

Increased mitoROS triggers the redistribution of eNOS and increases the binding between eNOS and Cav1, resulting in increased Cav1 SNO at C156. Increased Cav1 SNO reduces the interaction between Cav1 and cSrc, resulting in Cav1-cSrc dissociation and subsequent phosphorylation/activation of cSrc. Phosphorylated cSrc then competes with and displaces Cx43 from ZO-1 at the intercalated disc, leading to degradation of Cx43, conduction block, and increased propensity of ventricular arrhythmias

FIGS. 8A-D illustrate that there is no sodium current change or increased fibrosis in Cav1^{-/-} LV, according to one embodiment.

FIG. 8A illustrates the current-voltage curves of Na⁺ current (I_{Na}) densities in WT and Cav1^{-/-} LV myocytes (n=20 in each group). According to this embodiment, there were no significant differences in I_{Na} densities between WT and Cav1^{-/-} LV myocytes across the ranges of test potentials (−80 to 60 mV).

FIG. 8B shows that the steady state inactivation curves of I_{Na} of WT and Cav1^{-/-} LV myocytes were indistinguishable, according to this embodiment.

As shown in FIG. 8C and FIG. 8D, Mason trichrome staining of the LV cross-sections from WT and Cav1^{-/-} mice did not reveal evidence of increased fibrosis in Cav1^{-/-} LV.

FIG. 9 illustrates that the C-terminal Src kinase (CSK) does not co-immunoprecipitate with cSrc in mouse left ventricle (LV), according to one embodiment. In this embodiment, Immunoprecipitation with cSrc antibody using the protein lysates from WT and ACE8/8 LV did not show evidence of interaction between CSK and cSrc.

FIG. 10 illustrates that Nitric oxide (NO) production does not differ in WT and ACE8/8 ventricular cardiomyocytes in this embodiment. As illustrated in this figure, chemiluminescence nitric oxide measurements did not reveal significant differences in NO production from WT and ACES/8 ventricular cardiomyocytes.

### DETAILED DESCRIPTION

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

All patents and publications referred to herein are incorporated by reference in their entirety.

### CERTAIN EXEMPLARY TERMINOLOGY

Unless defined otherwise, all technical and scientific terms used herein have the same meaning as is commonly understood by one of skill in the art to which the claimed subject matter belongs. In the event that there is a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless otherwise stated. Furthermore, use of the term “including” as well as other forms, such as “include”, “includes,” and “included,” is not limiting.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety for any purpose. Abbreviations used herein have their conventional meaning within the chemical and biological arts.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the appended claims define the scope of the invention and that methods and structures within the scope of these claims and their equivalents be covered thereby.

Unless defined otherwise, all technical and scientific terms used herein have the same meaning as is commonly understood by one of skill in the art to which this invention belongs.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods and compositions described herein.

As used herein, “treatment” or “treating,” or “palliating” or “ameliorating” is used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. The treatment or amelioration of symptoms can be based on objective or subjective parameters; including the results of a physical examination, functional (self) evaluation, and/or any form of vision evaluation.

“Subject” refers to an animal, such as a mammal, for example a human. The methods described herein can be useful in both human therapeutics and veterinary applications. In some embodiments, the patient is a mammal, and in some embodiments, the patient is human.

The term “in vivo” refers to an event that takes place in a subject's body.

The term “in vitro” refers to an event that takes places outside of a subject's body. For example, an in vitro assay encompasses any assay run outside of a subject assay. In vitro assays encompass cell-based assays in which cells alive or dead are employed. In vitro assays also encompass a cell-free assay in which no intact cells are employed.

### General Discussion of Technology

Accumulating evidence has suggested that Cav1 is involved in the regulation of cardiac electrical functioning. For example, Cav1 binds to the human ether-a-go-go related gene (hERG) K⁺ channel and regulates its function and degradation. See, Lin et al, The regulation of the cardiac potassium channel (HERG) by caveolin-1, Biochem Cell Biol. 2008; 86:405-415; and Massaeli et al, Involvement of caveolin in low K⁺-induced endocytic degradation of cell-surface human ether-a-go-go-related gene (hERG) channels, J Biol Chem. 2010; 285:27259-27264.

L-type Ca²⁺ channels, as well as Cx43, have been shown to be targeted to lipid rafts/caveolae and directly interact with Cav1. See, Darby et al, Caveolae from canine airway smooth muscle contain the necessary components for a role in Ca²⁺ handling, Am J Physiol Lung Cell Mol Physiol. 2000, 279:L1226-1235; and Schubert et al, Connexin family members target to lipid raft domains and interact with caveolin-1, Biochemistry, 2002, 41:5754-5764.

Importantly, human genome-wide association studies have revealed significant association of Cav1 variants with increased risk of cardiac arrhythmias. See, Holm et al, Several common variants modulate heart rate, PR interval and QRS duration, Nat Genet. 2010, 42:117-122; and Ellinor et al., Meta-analysis identifies six new susceptibility loci for atrial fibrillation. Nat Genet. 2012; 44:670-675.

Using two different mouse models (Cav1^{-/-} and ACES/8) in the present study, the essential role of Cav1 in maintaining the homeostasis of cardiac Cx43 by modulating cSrc activity was demonstrated. With the abrogation of Cav1-mediated cSrc inhibition, either through genetic deletion of Cav1 or via Cav1 SNO induced by enhanced RAS signaling, cSrc became activated, leading to down-regulation of Cx43, reduced ventricular conduction velocity, and increased propensity for ventricular arrhythmias.

The renin-angiotensin system (RAS) is a critical component of the physiological and pathological responses of the cardiovascular system. Angiotensin II (AngII), the central signaling effector of RAS, binds to AngII type 1 receptor (AT1R) and activates NAD(P)H oxidases leading to increased production of cytosolic as well as mitochondrial ROS. See, Mollnau et al., Effects of angiotensin II infusion on the expression and function of NAD(P)H oxidase and components of nitric oxide/cGMP signaling, Circ Res. 2002, 90:E58-65; and Doughan et al., Molecular mechanisms of angiotensin II-mediated mitochondrial dysfunction: linking mitochondrial oxidative damage and vascular endothelial dysfunction, Circ Res. 2008, 102:488-496.

It has also been demonstrated that mitochondrial, but not cytosolic, ROS plays a critical role in RAS-mediated connexon remodeling and ventricular arrhythmias. See, Sovari et al., Mitochondria oxidative stress, connexin43 remodeling, and sudden arrhythmic death, Circ Arrhythm Electrophysiol 2013, 6:623-631.

Provided herein is a mechanistic link between RAS-induced oxidative stress and ventricular arrhythmias, where RAS-induced mitochondrial ROS triggers increased eNOS-Cav1 association and Cav1-S-nitrosation, resulting in cSrc activation, Cx43 degradation and subsequent electrical abnormalities.

Cav3 is the muscle-specific caveolin isoform that is essential for caveolae formation in cardiomyocytes. See, Woodman et al., Caveolin-3 knock-out mice develop a progressive cardiomyopathy and show hyperactivation of the p42/44 MAPK cascade, J Biol Chem. 2002, 277:38988-38997. Intriguingly, it was discovered that Cav3 was not involved in the regulation of cSrc and Cx43, since Cav3 did not interact with cSrc (see, FIG. 3C and FIG. 3D) and knockout of Cav3 did not alter cardiac cSrc activity or Cx43 expression levels (see, FIG. 2D).

The observation that cSrc is not activated in Cav3^{-/-} LV suggests that Cav1-mediated cSrc inhibition is unaffected in Cav3^{-/-} hearts. Because caveolae are completely absent in Cav3^{-/-} cardiomyocytes, (see, Woodman et al., Caveolin-3 knock-out mice develop a progressive cardiomyopathy and show hyperactivation of the p42/44 MAPK cascade, J Biol Chem. 2002, 277:38988-38997) the preserved Cav1-cSrc interaction in Cav3^{-/-} hearts suggests that Cav1 interacts with and regulates cSrc outside of caveolae in cardiomyocytes.

Indeed, recent studies indicate that caveolin can regulate cellular functions in non-caveolar regions. Examples include cell adhesion, reactive neuronal plasticity and oxidative stress-induced responses. See, del Pozo et al, Phospho-caveolin-1 mediates integrin-regulated membrane domain internalization, Nat Cell Biol. 2005, 7:901-908; Gaudreault et al, A role for caveolin-1 in post-injury reactive neuronal plasticity, J Neurochem. 2005, 92:831-839; and Khan et al., Epidermal growth factor receptor exposed to oxidative stress undergoes Src- and caveolin-1-dependent perinuclear trafficking, J Biol Chem. 2006, 281:14486-14493. Taken together, the data presented here provide evidence suggesting the non-caveolar role of Cav1-mediated cSrc and Cx43 regulation in cardiomyocytes.

Cav1 is known to negatively regulate eNOS activity in endothelial cells in a caveolae-dependent manner. See, Sowa et al., Distinction between signaling mechanisms in lipid rafts vs. caveolae, Proc Natl Acad Sci USA 2001, 98:14072-14077. In cells where Cav1 does not drive caveolae assembly, however, the ability of Cav1 to inhibit eNOS activity is diminished, albeit the Cav1-eNOS interaction remains. The observation that Cav1-eNOS binding increased without altering eNOS activity (levels of p-eNOS) in ACE8/8 cardiomyocytes (see, FIG. 5C and FIG. 5D) suggests that the Cav1-eNOS interaction in cardiomyocytes is non-caveolar.

Therefore, upon enhanced RAS activity and increased mitoROS, eNOS actively redistributes to non-caveolar compartments, allowing spatially confined NO release to targets such as Cav1. This observation highlights the importance of the spatial coupling and direct interaction between eNOS and its targets in NO-mediated signaling pathways. See, Nedvetsky et al., There's NO binding like NOS binding: protein-protein interactions in NO/cGMP signaling, Proc Natl Acad Sci USA 2002, 99:16510-16512.

In addition, the paradox that binding between eNOS and its negative regulator Cav1 in ACE8/8 mouse hearts allows nitrosation of Cav1 suggests that Cav1 may cease to inhibit eNOS if an appropriate signal is given. It is possible that upon an enhanced RAS state, the non-caveolar interaction between eNOS and Cav1 is increased, and this leads to potential increased local activity of eNOS to facilitate Cav1 SNO. The differential eNOS activities in caveolar and non-caveolar compartments also suggest that the lipid environment may contribute to the negative regulation of eNOS where eNOS targeted to non-caveolar regions can be activated even in the presence of Cav1. See, Michel et al., Nitric oxide synthases: which, where, how, and why? J Clin Invest. 1997, 100:2146-2152.

As discussed herein, it was discovered that increased eNOS-Cav1 binding upon RAS activation in cardiomyocytes was dependent on mitoROS. This is in line with the recent evidence showing that mitochondrial-targeted, but not general, antioxidants, can ameliorate RAS activation-induced Cx43 downregulation and ventricular arrhythmias. See, Sovari et al., Mitochondria oxidative stress, connexin43 remodeling, and sudden arrhythmic death, Circ Arrhythm Electrophysiol, 2013, 6:623-631. These findings reflect the critical role of mitoROS in cardiac cSrc and Cx43 regulation.

The data provided herein is also consistent with the emerging role of mitoROS as signaling molecules in regulating physiological functions including autophagy, differentiation, and adaptation to hypoxia. See, Qi et al., Bnip3 and AIF cooperate to induce apoptosis and cavitation during epithelial morphogenesis, J Cell Biol. 2012, 198:103-114; Tormos et al., Chandel NS. Mitochondrial complex III ROS regulate adipocyte differentiation, Cell Metab. 2011, 14:537-544; and Brunelle et al., Oxygen sensing requires mitochondrial ROS but not oxidative phosphorylation, Cell Metab. 2005, 1:409-414.

How mitoROS signals the redistribution of eNOS to non-caveolar Cav1, causes Cav1 SNO, and contributes to subsequent cSrc and Cx43 dysregulation is important to understand. It has been reported that a subpopulation of eNOS is “docked” to the mitochondrial outer membrane both in endothelial cells and neurons. It is possible that this subpopulation of eNOS senses the increased mitoROS upon RAS activation, resulting in its displacement from the mitochondria outer membrane and redistribution to non-caveolar compartments where eNOS-Cav1-cSrc interaction occurs.

In summary, for the first time the critical role of Cav1 in maintaining the homeostasis of cardiac Cx43 by interacting with and inhibiting cSrc tyrosine kinase has been demonstrated. The disrupted Cav1-cSrc interaction upon pathological conditions such as enhanced RAS signaling resulted in the activation of cSrc, Cx43 reduction, slow conduction and increased risk for ventricular arrhythmias.

As summarized in the schematic illustration provided in FIG. 7, the data discussed herein suggest that mitoROS production increases upon RAS activation, which triggers the redistribution of eNOS and increased Cav1-eNOS interaction, resulting in Cav1 SNO, Cav1-cSrc dissociation, cSrc activation, Cx43 degradation and subsequently, slow cardiac conduction and increased propensity for arrhythmias.

These findings provide a potential explanation for the genetic association of Cav1 and human arrhythmias, as well as the insights into the mechanistic link between RAS-induced mitochondrial ROS and Cx43 hemichannel regulation. These results provide a potential therapeutic approach of targeting the regulation of Cav1 or mitochondrial ROS to ameliorate arrhythmic risk caused by RAS activation in various cardiac diseases

### Exemplary Methods of Treatment

In various embodiments, gene therapy is used to increase or decrease Cav1 in a patient at risk of arrhythmia.

In some embodiments, a compound that inhibits NOS or improves NOS uncoupling (e.g., Tetrahydrobiopterin (THB) dihydrochloride, CAS 17528-72-2) is administered to a patient to decrease the patient's risk of arrhythmia. Exemplary eNOS inhibitors that useful in this embodiment of the invention include, but are not limited to, L-NG-Nitroarginine Methyl Ester (“L-NAME”; CAS 51298-62-5), N(5)-(1-Iminoethyl)-L-ornithine HCl (“L-NIO”; CAS 150403-88-6), or L-NG-Monomethylarginine, Acetate Salt (“L-NMMA”; CAS 53308-83-1).

In some embodiments, an antioxidant is administered to a patient to decrease Cav1 nitrosylation in the patient. In this embodiment, the decrease in Cav1 nitrosylation regulates Cav1 and decreases the patient's risk of arrhythmia. As used herein, an “antioxidant” is a compound/molecule that inhibits the oxidation of other molecules. In specific embodiments, the antioxidant competes for S-nitrosylation such as N-Acetyl-cystein (CAS 616-91-1) or glutathione or reduces mitochondrial oxidative stress in the cell.

Exemplary antioxidants useful in this embodiment of the invention include, but are not limited to, Nicotinamide adenine dinucleotide NAD⁺, Free Acid (CAS 53-84-9), (2-(2,2,6,6-Tetramethylpiperidin-1-oxyl-4-ylamino)-2-oxoethyl)triphenylphosphonium chloride monohydrate (“Mito-TEMPO”), DL-alpha-lipoic acid, manganese superoxide dismutase (“MnSOD”), or other mitochondrial targeted antioxidants. In some specific embodiments, a composition that alters manganese superoxide dismutase in the cells is administered to the patient.

Having now generally described the invention, the same will be more readily understood through reference to the following examples which are provided by way of illustration, and are not intended to be limiting of the present invention, unless specified.

### EXAMPLES

Statistical Analysis:

All averaged WB densitometry and LV conduction velocity measurements were presented as means±SEM. The inducibility of VT was presented as percentage of all tested animals in the same group. The statistical significance of differences between experimental groups was evaluated by Mann-Whitney U test or Fisher's exact test; P values <0.05 are considered statistically significant.

Experimental Animals:

Animals were handled in accordance with the NIH Guide for the Care and Use of Laboratory Animals. All protocols involving animals were approved by the Animal Studies Committee at the University of Illinois at Chicago, Lifespan, or the Veterans Administration San Diego Healthcare System. Experiments were performed on Cav1^{-/-}, Cav3^{-/-}and ACE8/8 mice (all in C57/Bl6 background) that were derived and maintained as described in: Xiao et al., Mice with cardiac-restricted angiotensin-converting enzyme (ACE) have atrial enlargement, cardiac arrhythmia, and sudden death, Am J Pathol. 2004, 165:1019-1032; Razani et al., Caveolin-1 null mice are viable but show evidence of hyperproliferative and vascular abnormalities, J Biol Chem. 2001, 276:38121-38138; and Hagiwara et al., Caveolin-3 deficiency causes muscle degeneration in mice, Hum Mol Genet. 2000, 9:3047-3054.

Western Blotting:

For Western blots, total protein lysates were prepared from the LV of 6 week-old WT control, ACE8/8 with and without 2 week treatment of mitochondria-targeted antioxidant (2-(2,2,6,6-Tetra-methylpiperidin-1-oxyl-4-ylamino)-2-oxoethyl)-triphenylphosphonium chloride (MitoTEMPO, see below), as well as from adult (2-4 month) Cav1−/− mice with and without 4 weeks treatment of cSrc inhibitor 1-(1,1-dimethylethyl)-1-(4-methylphenyl)-1H-pyrazolo[3,4-d]pyrimidin-4-amine (PP1, see below).

In some cases, protein lysates were prepared from the LV cardiomyocytes isolated from ACE8/8 animals using described methods. 13 Total protein lysates were fractionated on 8-15% SDS-PAGE and transferred to PVDF membranes, incubated in 5% skim milk in PBS containing 0.1% Tween 20 (blocking buffer) for 1 h at room temperature, followed by overnight incubation at 4° C. with primary antibodies (rabbit monoclonal anti-cSrc, p-cSrc at Tyr416, Cx43, C-terminal Src kinase [CSK] and Tyr14 p-Cav1 antibodies from Cell Signaling, mouse monoclonal anti-Cav1 and Cav3 antibodies from BD Biosciences, rabbit monoclonal anti-eNOS, p-eNOS and nNOS antibodies from Santa Cruz).

For a loading control, the membranes were blotted with primary antibodies against glyceraldehydes-3-phosphate dehydrogenase (GAPDH) (Santa Cruz Biotech, Santa Cruz, Calif.). After washing, the membranes were incubated for 1 h at room temperature with alkaline phosphatase-conjugated secondary antibody diluted in blocking buffer, and bound antibodies were detected using a chemiluminescent alkaline phosphate substrate. Protein band intensities were quantified by densitometry (Quantity One Basic, Bio-Rad Laboratory, Hercules, Calif.) and the band densities of each protein in individual samples were normalized to that of GAPDH in the same sample.

### Example 1

### Immunoprecipitation of Cav1, Cav3 and cSrc

Immunoprecipitation (IP) of Cav1 was conducted using a magnetic IP kit from Thermo Scientific (Waltham, Mass.). Protein lysates from total LV or isolated LV myocytes (with 1000 μg total protein) from control and ACES/8 mice were incubated with 10 μg of mouse anti-Cav1 monoclonal antibodies overnight at 4° C.

The immune complex was bound to protein A/G magnetic beads and collected with a magnetic stand. Proteins co-immunoprecipitated with Cav1 were eluted and subjected to gel electrophoresis and Western blotting using the antibodies described above where appropriate. The amount of proteins co-immunoprecipitated with Cav1 was normalized to total Cav1 co-immunoprecipitated in each sample. Similar methods were used to analyze the proteins that co-immunoprecipitated with Cav3 and cSrc using antibodies against Cav3 (mouse monoclonal, BD Biosciences, San Jose, Calif.) or cSrc (rabbit monoclonal, Cell Signaling Technology, Danvers, Mass.).

### Example 2

### Generation of Cav1 Cystein-to-Serine Mutants and Transfection

A full-length mouse Cav1 cDNA clone in pCMV-SPORT6 vector was acquired from Thermo Scientific (MGC mouse Cav1 cDNA, clone ID 4484857). The cysteine-to-serine Cav1 mutants (C133S, C143S and C156S) were generated from this WT mouse Cav1 clone using the QuickChange II Site-Directed Mutagenesis kit (Agilent Technologies) according to the manufacturer's instructions.

The primer sequences used for generation of these Cav1 mutant clones were:

C133S:
sense
5′-gggcggttgtaccgagcatcaagagcttc-3′
anti-sense
5′-gaagctcttgatgctcggtacaaccgccc-3′
C143S:
sense
5′-cctgattgagattcagagcatcagccgcgtcta-3′
anti-sense
5′-tagacgcggctgatgctctgaatctcaatcagg-3′
C156S:
sense
5′-tctacgtccataccttcagcgatccactctttgaa-3′
anti-sense
5′-ttcaaagagtggatcgctgaaggtatggacgtaga-3′

Transfection of HEK cells with designated plasmids was conducted using Lipofectamine 2000 according to manufacturer's protocol.

### Example 3

### Detection of Cav1 S-Nitrosation

S-nitrosated Cav1 was detected in cells (isolated ventricular cardiomyocytes from control and ACES/8 mice or HEK cells transfected with WT Cav1 or Cav1 mutants [C156S, C143S or C133S], with or without NO donor, SNAP [20 μM, 10 min], treatment) using the methods described in Jaffrey et al., Protein S-nitrosylation: a physiological signal for neuronal nitric oxide, Nat Cell Biol. 2001, 3:193-197; and Haendeler et al., Redox regulatory and anti-apoptotic functions of thioredoxin depend on S-nitrosylation at cysteine 69, Nat Cell Biol. 2002, 4:743-749.

Cells were lysed with HENS buffer (25 mM HEPES, pH 7.7, 0.1 mM EDTA, 0.01 mM neocuproine and 1% SDS) and centrifuged at 20,000 g for 15 min. The total cell protein was incubated in 20 mM methylmethanthiosulphate (MMTS) for 20 min at 50° C. and vortexed for 5 s every 2 min. Cellular protein was precipitated with acetone.

After removing acetone, protein pellet was resuspended in HENS buffer. N-[6-(biotinamido)hexyl]-3′-(2′-pyridyldithio)propionamide (biotin-HPDP, 400 μM) and sodium ascorbate (1 mM) was added for 1 h at 25° C. in the dark. Cav1 was then immunoprecipitated from each sample using monoclonal Cav1 antibody (BD Biosciences, San Jose, Calif.), where S-nitrosated Cav1 was detected by HRP-conjugated streptavidin following gel electrophoresis and Western blotting. The amount of S-nitrosated Cav1 was quantified and normalized to total Cav1 in each sample

### Example 4

### Ventricular Conduction Velocity Measurement

LV conduction velocity was measured in anesthetized WT (n=6) and Cav1−/− (with and without 4 week PP1 treatment, n=6 in each group) mice using a flexible multielectrode array (Flex-MEA, 72 electrodes) system (Multichannel systems, Reutlingen, Germany) according to manufacturer's instructions.

Mid-anterior LV epicardial electrical propagation was recorded under right-ventricular pacing (750 bpm); the color mapping of LV conduction propagation, as well as the calculation of LV conduction velocity, were carried out using Cardio 2D software (Multichannel systems, Reutlingen, Germany)

### Example 5

### Surface Electrocardiogram Recording and Programmed Ventricular Stimulation

Surface electrocardiograms (ECG) were recorded and ventricular arrhythmia inducibility was determined in WT and Cav1^{-/-} with and without 4 weeks of PP1 treatment (n=4-6 in each group) using described methods under general anesthesia with isofurane as described in Berul et al., In vivo cardiac electrophysiology studies in the mouse, Circulation 1996, 94:2641-2648.

Surface electrocardiograms (ECG) were monitored and recorded with needle electrodes connected to a dual bioamplifier (PowerLab 26T, AD Instruments, Dunedin, New Zealand) as described in Yang et al., Exercise training and PI3Kα-induced electrical remodeling is independent of cellular hypertrophy and Akt signaling, J Mol Cell Cardiol. 2012, 53:532-541.

Baseline ECG was acquired for 2 minutes; the data was stored and subsequently analyzed offline using the LabChart 7.1 (AD Instrument) software. Lead II recordings were chosen for analyses. The measurement is illustrated in FIG. 1A. QT intervals were corrected for heart rate using the formula QTc=QT/(√RR/100). See, Mitchell et al., Measurement of heart rate and Q-T interval in the conscious mouse, Am J Physiol. 1998, 274:H747-751.

Programmed ventricular stimulation was performed with a RV epicardial electrode connected to STG1008 stimulator (Multichannel systems, Reutlingen, Germany), where eight consecutive beats were paced at 60 ms basic cycle length, followed by triple extrastimuli with incrementally deceasing cycle lengths between 20-55 ms, and inducible ventricular tachycardia was defined as >3 consecutive ventricular beats. See, Bevilacqua et al., A targeted disruption in connexin40 leads to distinct atrioventricular conduction defects, J Interv Card Electrophysiol. 2000, 4:459-467.

### Example 6

### Measurement of Nitric Oxide (NO) Production by Chemiluminescence

Isolated LV cardiomyocytes from WT and ACES/8 mice were plated in 6-well plates. After adherence, myocytes were washed twice with HBSS and incubated with serum free DMEM or HBSS at 37° C. for one hour. After incubation, medium was collected and centrifuged shortly to remove floating cells. NO concentration in the culture media was assessed by measuring NO₂⁻ accumulation using a Sievers 280i Nitric Oxide Analyzer (Sievers Instruments, Boulder, Colo.).

NO production was assessed from accumulated NO₂⁻ level in the media and reported as nmol NO per mg protein. A standard curve was generated using authentic sodium nitrite (NaNO₂) for calibration.

### Example 7

### Loss of Cav1 Results in Slowed Cardiac Conduction and Increased Risk of Ventricular Arrhythmia

To determine the potential impact of genetic deletion of Cav1 on cardiac electric functioning, adult (2-4 months) WT and Cav1^{-/-} mice were first subjected to surface ECG recordings (FIG. 1A).

Cav1^{-/-} mice were viable and fertile without evidence of cardiac structural abnormality up to 5 months of age. See, Razani et al., Caveolin-1 null mice are viable but show evidence of hyperproliferative and vascular abnormalities, J Biol Chem. 2001, 276:38121-38138.

The ECG recordings revealed that the morphologies of the P, J and T waves, as well as the durations of the PR, QRS, and corrected QT (QTc) intervals (FIG. 1D) measured in WT and Cav1^{-/-} animals were indistinguishable, although the R wave amplitudes were trending lower in Cav1^{-/-} compared with WT mice (FIG. 1A and FIG. 1B).

Using a 72-electrode Flex-MEA, the LV epicardial conduction velocity was measured in WT and Cav1^{-/-} mice. As shown in FIG. 1C, the LV conduction velocity in Cav1^{-/-} (n=6, 0.35±0.03 mm/ms) was significantly (P<0.05) lower than that in WT (n=6, 0.50±0.09 mm/ms) mice.

To test if the reduced LV conduction velocity observed in Cav1^{-/-} mice was associated with increased arrhythmia risk, epicardial programmed electrical stimulation was conducted in WT and Cav1^{-/-} mice, which revealed that non-sustained ventricular tachycardia (NSVT) could be induced in 75% (n=4) of Cav1 mice, whereas none (n=6) of the WT mice were inducible (P<0.05 by Fisher's exact test, FIG. 1D).

Taken together, initial electrophysiological studies demonstrated that loss of Cav1 resulted in slowed LV conduction velocity and increased ventricular arrhythmia inducibility

### Example 8

### Electrical Abnormalities Observed in CAv1^{-/-} Mice Result from LV Cx43 Down Regulation by Activated cSrc Tyrosine Kinase

Slow myocardial conduction velocity can result from reduced Na⁺ current (I_{Na}) or from increased cell-cell conduction resistance caused by increased fibrosis or decreased gap junction function. See, King et al., Determinants of myocardial conduction velocity: implications for arrhythmogenesis, Front Physiol. 2013, 4:154.

Whole-cell voltage clamp experiments in LV cardiomyocytes, as well as Mason-trichrome staining of the LV cross-sections, were conducted in WT and Cav1^{-/-} mice to determine if changes in I_{Na} currents or the presence of cardiac fibrosis may contribute to the conduction abnormality and arrhythmia phenotype observed in Cav1^{-/-} mice.

As shown in FIG. 8A and FIG. 8B, the densities of I_{Na}, as well as the steady state inactivation properties of I_{Na}, were similar in WT and Cav1^{-/-} LV myocytes. Also similar to WT LV, there was no significant fibrosis detected in Cav1^{-/-} LV (see, FIG. 8C and FIG. 8D). In contrast, Western blot analyses revealed a 42% reduction of the Cx43 expression in Cav1^{-/-}, compared with WT LV (FIG. 2A and FIG. 2B).

Isolated myocytes from WT and Cav1^{-/-} LV were used in additional Western blots to confirm that Cx43 expression levels were markedly reduced in Cav1^{-/-} compared to WT LV cardiomyocytes (52% reduction, P<0.001; FIG. 2C). Taken together, this data suggests that the conduction abnormality and increased inducibility for ventricular arrhythmias observed in Cav1^{-/-} mice can be attributed largely to Cx43 downregulation.

It is known that Cav1 negatively regulates a redox-sensitive tyrosine kinase cSrc, the activation of which has been shown to cause the downregulation of cardiac Cx43. See, Kieken et al., Structural and molecular mechanisms of gap junction remodeling in epicardial border zone myocytes following myocardial infarction, Circ Res. 2009, 104:1103-1112.

We hypothesized that the observed Cx43 downregulation, slow conduction and increased arrhythmic inducibility in Cav1^{-/-} mice resulted from loss of Cav1 inhibition of cSrc. To test this, the expression levels of phosphorylated cSrc at Tyr⁴¹⁶ (p-cSrc, the active form of cSrc) in the ventricular myocardium and isolated LV cardiomyocytes from WT and Cav1^{-/-} mice were examined. As shown in FIGS. 2A and 2C, the protein expression level of p-cSrc was significantly upregulated in Cav1^{-/-} LV (by 2.8 fold, P<0.001) and isolated LV cardiomyocytes (by 2.5 fold, P<0.001), compared to WT.

In addition, pharmacological inhibition of cSrc activity with 4 weeks of the cSrc inhibitor PP1 (1.5 mg/kg/dose, 3 times per week for 4 weeks, intraperitoneally) in Cav1^{-/-} mice normalized LV p-cSrc and Cx43 expression to levels similar to that in WT (FIG. 2A and FIG. 2B). Consistent with the reversal of Cx43 downregulation with cSrc inhibition, the slow LV conduction and increased ventricular arrhythmia inducibility observed in Cav1 mice could be mitigated by 4-week treatment with cSrc inhibitor PP1 (LV conduction velocity 0.43±0.01 mm/ms; 0% inducible for VT, n=6, FIGS. 1C and 1D). In contrast to Cav1^{-/-} mice, the LV p-cSrc and Cx43 expression in Cav3^{-/-} LV were similar to that in WT (FIG. 2D), suggesting no role of Cav3 in cSrc/Cx43 regulation.

Taken together, these results suggest that Cav1, but not Cav3, plays a critical role in maintaining cardiac Cx43 homeostasis through regulating cSrc activity. In the absence of Cav1, cSrc becomes activated, leading to Cx43 downregulation, subsequent conduction abnormality, and increased risk for arrhythmias

### Example 9

### Reduced Binding Between Cav1 and cSrc Results in cSrc Activation and Subsequent Cx43 Down-Regulation Upon Enhanced Cardiac RAS Signaling

The electrophysiological abnormalities linked to Cx43 dysregulation observed in Cav1^{-/-} mice were reminiscent of the phenotype of the mouse models with increased cardiac RAS activity. See, Xiao et al., Mice with cardiac-restricted angiotensin-converting enzyme (ACE) have atrial enlargement, cardiac arrhythmia, and sudden death, Am J Pathol. 2004, 165:1019-1032; and Donoghue et al., Heart block, ventricular tachycardia, and sudden death in ACE2 transgenic mice with downregulated connexins, JMol Cell Cardiol. 2003, 35:1043-1053. These animals have a high incidence of conduction block, ventricular arrhythmias and sudden death resulting from reduced cardiac Cx43 and impaired gap junction function.

Using a gene-targeted mouse model of cardiac-specific ACE overexpression (ACES/8), it was demonstrated that enhanced cardiac RAS signaling can lead to cSrc activation, Cx43 degradation, reduce myocyte coupling, increased inducibility of ventricular arrhythmias and sudden cardiac death, all of which can be reversed by pharmacological inhibition of cSrc. See, Xiao et al., Mice with cardiac-restricted angiotensin-converting enzyme (ACE) have atrial enlargement, cardiac arrhythmia, and sudden death, Am J Pathol. 2004, 165:1019-1032; and Sovari et al., Inhibition of c-Src tyrosine kinase prevents angiotensin II-mediated connexin-43 remodeling and sudden cardiac death, J Am Coll Cardiol. 2011, 58:2332-2339.

Given the similarity in the electrophysiological phenotypes of Cav1^{-/-} and ACE8/8 mice, we hypothesized that Cav1 was likely involved in RAS-induced cardiac cSrc activation and Cx43 reduction.

Increased cardiac RAS activity in ACE8/8 mice was accompanied by a 3.5 fold increase (P<0.001) in cSrc activation/phosphorylation and 77% reduction in Cx43 (P<0.001) compared to WT LV (FIG. 3A and FIG. 3B). The intrinsic kinase activity of cSrc is controlled by autophosphorylation of Tyr⁴¹⁶ located within the kinase domain that results in cSrc activation and by phosphorylation at Tyr⁵²⁷ that result in cSrc inactivation. See, Brown et al., Regulation, substrates and functions of src, Biochim Biophys Acta. 1996, 1287:121-149.

Phosphorylation of Tyr⁵²⁷ is mediated by the C-terminal Src kinase (CSK), whereas cSrc Tyr⁴¹⁶ autophosphorylation can be suppressed by the direct binding with the scaffolding proteins Cav1 and Cav3. See, Okada et al., CSK: a protein-tyrosine kinase involved in regulation of Src family kinases, J Biol Chem. 1991, 266:24249-24252; Place et al., Cooperative role of caveolin-1 and C-terminal Src kinase binding protein in C-terminal Src kinase-mediated negative regulation of c-Src, Mot Pharmacol. 2011, 80:665-672; and Li et al., tyrosine kinases, Galpha subunits, and H-Ras share a common membrane-anchored scaffolding protein, caveolin. Caveolin binding negatively regulates the auto-activation of Src tyrosine kinases, J Biol Chem. 1996, 271:29182-29190.

Cav1 is also necessary for CSK recruitment to cSrc. See, Patel et al., Mechanisms of cardiac protection from ischemia/reperfusion injury: a role for caveolae and caveolin-1, FASEB J. 2007, 21:1565-1574. We hypothesized that enhanced RAS signaling activated cSrc either through decreasing the availability of the negative regulator(s) or through abrogating the interaction between cSrc and its negative regulator(s). To test this, the protein expression levels of CSK, Cav3, Cav1, as well as phosphorylated Cav1 (at Tyr¹⁴), the active form of Cav1 shown to inhibit cSrc activity (see, Okada et al., CSK: a protein-tyrosine kinase involved in regulation of Src family kinases. J Biol Chem. 1991, 266:24249-24252), were examined and compared in WT and ACE8/8 LV samples.

As shown in FIGS. 3A and 3B, the protein expression of cSrc negative regulators, CSK, Cav3 and Cav1/p-Cav1, were not significantly different in WT and ACE8/8 LV. Next, the interaction between cSrc and its negative regulators in the mouse LV was assessed.

Interestingly, cSrc failed to co-immunoprecipitate with CSK (see, FIG. 9) or Cav3 (FIG. 3C and FIG. 3D), whereas cSrc co-immunoprecipitated with Cav1 in mouse LV (FIG. 3E). In addition, the interaction between cSrc and Cav1 was markedly reduced (by 50%, P<0.001) in ACE8/8 compared with WT LV (FIG. 3E and FIG. 3F).

Taken together, these results suggest that reduced interaction between Cav1 and cSrc abrogates the inhibitory effects of Cav1 on cSrc, thereby contributing to cSrc activation upon enhanced RAS signaling in mouse ventricular myocardium

### Example 10

### Enhanced RAS Signaling Increases S-Nitrosation of Cav1, Resulting in Reduced CAv1-cSrc Interaction in LV Cardiomyocytes

It is known that the interaction between Cav1 and cSrc at the cell membrane depends on the coupling between the N-terminal myristoyl moiety of cSrc and the palmitoylated Cys¹⁵⁶ of Cav1. See, Lee et al., Palmitoylation of caveolin-1 at a single site (Cys-156) controls its coupling to the c-Src tyrosine kinase: targeting of dually acylated molecules (GPI-linked, transmembrane, or cytoplasmic) to caveolae effectively uncouples c-Src and caveolin-1 (TYR-14), J Biol Chem. 2001, 276:35150-35158.

Protein palmitoylation can be disrupted by nitrosation of cysteine residues (S-nitrosation, SNO) by direct competition for cysteine or by the displacement of palmitate; SNO cysteine modification is known to modulate the activity of various signaling molecules including PSD-95, β-adrenergic receptor and Cav1. See, Salaun et al., The intracellular dynamic of protein palmitoylation, J Cell Biol. 2010, 191:1229-1238; Ho et al., S-nitrosylation and S-palmitoylation reciprocally regulate synaptic targeting of PSD-95, Neuron. 2011, 71:131-141; Adam et al., Nitric oxide modulates β2-adrenergic receptor palmitoylation and signaling, J Biol Chem. 1999, 274:26337-26343; and Baker et al., S-Nitrosocysteine increases palmitate turnover on Ha-Ras in NIH 3T3 cells, J Biol Chem. 2000, 275:22037-22047.

We hypothesized that increased SNO of Cav1 may contribute to the observed uncoupling of cardiac Cav1 and cSrc upon enhanced RAS signaling. To test this hypothesis directly, a biotin-switch assay to detect protein SNO was conducted using isolated cardiomyocytes from WT and ACE8/8 LV As shown in FIG. 4A, there was a 5.5 fold increase (P<0.01) of Cav1 SNO in isolated myocytes from ACE8/8, compared to WT LV. The increased Cav1 SNO with increased RAS activity was accompanied by a 50% reduction (P<0.01) in Cav1-cSrc interaction in ACE8/8 compared with WT LV myocytes (FIG. 4B).

To test if increased Cav1 SNO could result in Cav1-cSrc dissociation, human embryonic kidney (HEK) cells transfected with mouse Cav1 and cSrc were treated with 20 μM nitric oxide (NO) donor S-nitroso-N-acetyl-DL-penicillamine (SNAP) or vehicle for 10 min. Increased Cav1 SNO induced by SNAP treatment resulted in decreased (by 58% compared to control, P<0.01) Cav1-cSrc binding (FIG. 4C), suggesting that increased Cav1 SNO directly disrupted the Cav1-cSrc interaction.

### Example 11

### Cys¹⁵⁶, but not Cys¹³³, is Critical for Cav1 S-Nitrosation

Cav1 contains three cysteines (C¹³³, C¹⁴³ and C¹⁵⁶) that can be palmitoylated, tethering Cav1 to the plasma membrane (FIG. 5A). Because protein S-nitrosation, like phosphorylation, usually occurs in the presence of conserved motifs in the primary amino acid sequence, we examined the amino acid sequences surrounding the cysteine residues of Cav1 to identify potential sites for S-nitrosation. Of the three cysteines present in Cav1, only Cys¹⁵⁶ resides within a consensus motif (G,S,T,C,Y,N,Q)(K,R,H,D,E)C(D,E) for S-nitrosation (FIG. 5A). Thus, Cys¹⁵⁶ is the Cav1 SNO site.

To confirm this, HEK cells transfected either with WT Cav1 or one of the nitrosation-resistant Cys-to-Ser (C¹³³S, C¹⁴³S or C¹⁵⁶S) Cav1 mutants were treated with SNAP (20 μM, 10 min) and assayed for Cav1 SNO by biotin-switch assay. As shown in FIG. 5B, SNAP treatment increased S-nitrosation in WT, C¹³³S- and C¹⁴³S-Cav1, but not in C¹⁵⁶S-Cav1, suggesting Cys¹⁵⁶ was the critical cysteine residue required for Cav1 SNO.

### Example 12

### Cardiac Cav1 S-Nitrosation Upon Enhanced RAS Signaling is Facilitated by Increased eNOS-Cav1 Association

Physiologically, the chemical reaction of protein S-nitrosation is favored upon increased availability of NO, either through increased NO production or by close proximity to the enzymes that synthesize NO, NO synthase (NOS). See, Hess et al., Protein S-nitrosylation: purview and parameters, Nat Rev Mol Cell Biol. 2005, 6:150-166; and Brenman et al., Interaction of nitric oxide synthase with the postsynaptic density protein PSD-95 and al-syntrophin mediated by PDZ domains, Cell 1996, 84:757-767.

To test if the increased Cav1 SNO upon enhanced cardiac RAS signaling was the result of elevated NO production, the protein expression levels of NOS in isolated LV cardiomyocytes from WT and ACE8/8 animals were examined. As shown in FIG. 5C, the protein expression levels of neuronal (nNOS) and endothelial (eNOS) NOS, as well as phospho-eNOS, the active form of eNOS, were not significantly different in WT and ACE8/8 cardiomyocytes.

In addition, a direct quantification of NO concentration did not reveal a measurable difference in NO production from isolated WT and ACE8/8 ventricular cardiomyocytes (FIG. 10).

To test if enhanced RAS signaling made NO available to Cav1 by bringing NOS in proximity to Cav1, the amount of NOS that could be co-immunoprecipitated with Cav1 in WT and ACE8/8 LV myocytes was examined. As shown in FIG. 5D, Western blots of the Cav1-pull down lysates revealed a 2.2-fold increase (P<0.05) in the binding between eNOS and Cav1 in ACE8/8, compared with WT isolated LV myocytes, nNOS, however, did not co-immunoprecipitate with Cav1 in either WT or ACE8/8 LV myocytes (data not shown).

Taken together, this data suggest that increased Cav1 SNO with enhanced cardiac RAS signaling is related to increased Cav1-eNOS binding

### Example 13

### Cardiac RAS-Induced eNOS-Cav1 Association is Dependent on Increased Mitochondrial ROS

Using the same ACE8/8 mouse model, it was demonstrated that cardiac ROS, specifically mitochondrial ROS (mitoROS), is markedly increased with enhanced RAS signaling. See, Sovari et al., Inhibition of c-Src tyrosine kinase prevents angiotensin II-mediated connexin-43 remodeling and sudden cardiac death, J Am Coll Cardiol. 2011, 58:2332-2339; and Sovari et al., Mitochondria oxidative stress, connexin43 remodeling, and sudden arrhythmic death, Circ Arrhythm Electrophysiol, 013; 6:623-631.

Treatment with mitochondria-targeted antioxidant MitoTEMPO, but not the other types of antioxidants, restores the Cx43 expression, normalizes gap junction conduction, as well as ameliorates ventricular arrhythmias and sudden cardiac death in ACE8/8 mice. See, Sovari et al., Mitochondria oxidative stress, connexin43 remodeling, and sudden arrhythmic death, Circ Arrhythm Electrophysiol. 2013, 6:623-631.

We hypothesized that increased mitoROS upon enhanced RAS signaling mediated Cx43 degradation through modulating the Cav1-cSrc interaction and cSrc activity. To test this, 4 week ACE8/8 animals were treated with MitoTEMPO (0.7 mg/kg/day, intraperitoneally) for 2 weeks, a regimen that normalizes elevated mitoROS in ACE8/8 hearts to the levels similar to WT controls. As shown in FIG. 6A and consistent with previous results, MitoTEMPO treatment in ACE8/8 mice resulted in reduced cardiac cSrc phosphorylation (by 63%, P<0.05) and increased Cx43 expression (by 1.9 fold, P<0.001) compared to untreated ACE8/8 animals.

Importantly, co-immunoprecipitation experiments revealed that the increased Cav1-eNOS binding and decreased Cav1-cSrc interaction observed in ACE8/8 LV were both reversed with the treatment of MitoTEMPO (FIG. 6B), suggesting that the increased Cav1-eNOS binding and subsequent Cav1-cSrc dissociation upon enhanced RAS signaling were dependent on mitochondrial ROS.

What is claimed is:

1. A method for reducing arrhythmic risk in a patient by regulating Cav1 in the patient.

2. The method of claim 1, wherein the regulation of Cav1 includes a decrease in Cav1 nitrosylation.

3. A method for reducing arrhythmic risk in a patient by administering a compound that inhibits NOS.

4. A method for reducing arrhythmic risk in a patient by administering a compound that improves NOS uncoupling in the patient.

5. A method for reducing arrhythmic risk in a patient by administering a composition that alters manganese superoxide dismutase in the cells of the patient.
